(19) United States
(12) Patent Application Publication
(10) Pub. No.: US 2024/0144469 A1
(43) Pub. Date: May 2, 2024

(54) SYSTEMS AND METHODS FOR AUTOMATIC CARDIAC IMAGE ANALYSIS

(71) Applicant: Shanghai United Imaging Intelligence Co., Ltd., Shanghai (CN)

(72) Inventors: Xiao Chen, Lexington, MA (US); Shanhui Sun, Lexington, MA (US); Terrence Chen, Lexington, MA (US); Arun Innanje, Lexington, MA (US)

(73) Assignee: Shanghai United Imaging Intelligence Co., Ltd., Shanghai (CN)

(21) Appl. No.: 17/973,982

(22) Filed: Oct. 26, 2022

(51) Int. Cl. G06T 7/00; G06T 7/11; G06T 7/30; G06V 10/764

(52) U.S. Cl. CPC G06T 7/0012; G06T 7/11; G06T 7/30; G06V 10/764; G06T 2207/10088; G06T 2207/30048

(57) ABSTRACT

Cardiac images such as cardiac magnetic resonance (CMR) images and tissue characterization maps (e.g., T1/T2 maps) may be analyzed automatically using machine learning (ML) techniques, and reports may be generated to summarize the analysis. The ML techniques may include training one or more of an image classification model, a heart segmentation model, or a cardiac pathology detection model to automate the image analysis and/or reporting process. The image classification model may be capable of grouping the cardiac images into different categories, the heart segmentation model may be capable of delineating different anatomical regions of the heart, and the pathology detection model may be capable of detecting a medical abnormality in one or more of the anatomical regions based on tissue patterns or parameters automatically recognized by the detection model. Image registration that compensates for the impact of motions or movements may also be conducted automatically using ML techniques.

### BACKGROUND

Cardiac magnetic resonance (CMR) is an advanced medical imaging tool that may enable non-invasive heart disease diagnosis and prevention. For example, CMR with late gadolinium enhancement (LGE) may be used to detect the presence of scar tissues, while T1 mapping, T2 mapping, and extracellular volume fraction (ECV) mapping may be used to detect edema, interstitial space changes, and/or lipid or iron overloads. Current methods for analyzing CMR images are manual in nature and, as such, time consuming and error-prone, preventing the use of CMR from reaching its full potential.

### SUMMARY

Described herein are systems, methods, and instrumentalities associated with automatic cardia image processing. In embodiments of the present disclosure, an apparatus capable of performing the image processing task may comprise at least one processor configured to obtain a plurality of medical images associated with a heart, and classify, based on a machine-learned image classification model, the plurality of medical images into multiple groups, wherein the multiple groups may include at least a first group comprising one or more short-axis images of the heart and a second group comprising one or more long-axis images of the heart. The processor may be further configured to process at least one group of medical images from the multiple groups, wherein, during the processing, the at least one processor may be configured to segment, based on a machine-learned heart segmentation model, the heart in one or more medical images into multiple anatomical regions, determine whether a medical abnormality exists in at least one of the multiple anatomical regions, and provide an indication of the determination (e.g., in the form of a report, a segmentation mask, etc.).

In embodiments of the present disclosure, the plurality of medical images of the heart may include at least one of a magnetic resonance (MR) image of the heart or a tissue characterization map of the heart such as a T1 map or a T2 map, and the apparatus may be configured to supplement the processing of one type of images with the other. In embodiments of the present disclosure, the apparatus may be configured to classify the plurality of medical images by detecting, based on the machine-learned image classification model, one or more anatomical landmarks (e.g., an mitral annulus and/or an apical tip) in an image, and determining the classification of the image based on the detected landmarks (e.g., the presence of the mitral annulus and/or apical tip may indicate that the image is a short axis image).

In embodiments of the present disclosure, the machine-learned heart segmentation model may be capable of delineating the chambers of the heart such as the left ventricle (LV) and the right ventricle (RV), or segmenting the heart into multiple myocardial segments including, e.g., one or more basal segments, one or more mid-cavity segments, and one or more apical segments. The segmentation may be conducted based on one or more anatomical landmarks detected by the machine-learned heart segmentation model such as the areas where the left ventricle of the heart intersects with the right ventricle of the heart.

In embodiments of the present disclosure, the at least one processor of the apparatus may be configured to determine a tissue pattern or tissue parameter associated with the at least one of the multiple anatomical regions, and further determine whether the medical abnormality exists in the at least one anatomical region of the heart based on the determined tissue pattern or tissue parameter. The tissue pattern or tissue parameter may be determined based on a machine-learned pathology detection model trained for such purposes, in which case the machine-learned pathology detection model may be further trained to segment the area of the heart that is associated with the tissue pattern or tissue parameter from the corresponding anatomical region (e.g., via a segmentation mask, a bounding box, etc.).

In embodiments of the present disclosure, the at least one processor of the apparatus may be further configured to register two or more medical images of the heart (e.g., a cine image and a tissue characterization map) based on a machine-learned image registration model, wherein the image registration model may be trained to compensate for a motion associated with the two or more medical images during the registration. The registered images may then be used together to perform a comprehensive analysis of the heart's healthy state.

### BRIEF DESCRIPTION OF THE DRAWINGS

A more detailed understanding of the examples disclosed herein may be had from the following description, given by way of example in conjunction with the accompanying drawings.

FIG. 1 is simplified diagram illustrating an example of a machine learning based system or apparatus for automatically processing cardiac images such as CMR images and/or tissue characterization maps, according to some embodiments described herein.

FIG. 2 is a simplified diagram illustrating an example of an artificial neural network that may be used to implement and/or learn an image classification model, according to some embodiments described herein.

FIG. 3 is simplified diagram illustrating an example of an artificial neural network that may be used to implement and/or learn a heart segmentation model, according to some embodiments described herein.

FIG. 4A is a simplified diagram illustrating examples of heart segments (e.g., segments of a myocardium) that may be identified using a machine-learned heart segmentation model, according to some embodiments described herein.

FIG. 4B is a simplified diagram illustrating an example of a bullseye plot that may be used to report cardiac parameters determined using machine learning techniques, according to some embodiments described herein.

FIG. 4C a simplified diagram illustrating an example of myocardium segmentation, according to some embodiments described herein.

FIG. 5 is a simplified diagram illustrating an example of cardiac pathology determination using machine learning techniques, according to some embodiments described herein.

FIG. 6 is a flow diagram illustrating an example method for training a neural network to perform one or more of the tasks as described with respect to some embodiments provided herein.

FIG. 7 is a simplified block diagram illustrating an example system or apparatus for performing one or more of the tasks as described with respect to some embodiments provided herein.

### DETAILED DESCRIPTION

The present disclosure is illustrated by way of example, and not by way of limitation, in the figures of the accompanying drawings. A detailed description of illustrative embodiments will now be described with reference to the various figures. Although this description provides a detailed example of possible implementations, it should be noted that the details are intended to be exemplary and in no way limit the scope of the application.

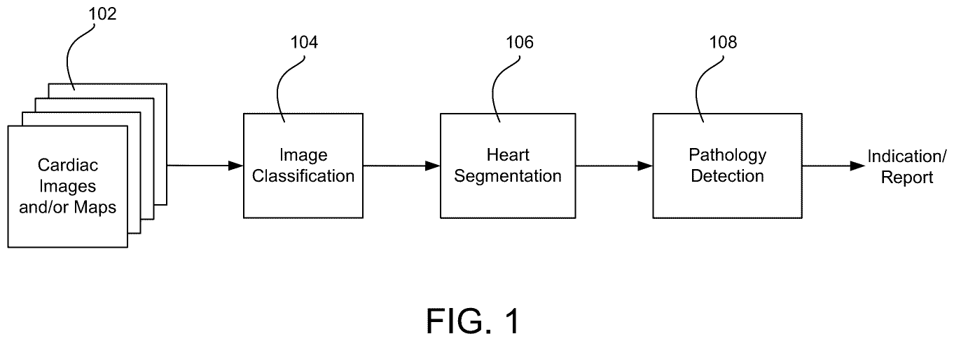

FIG. 1 is a block diagram illustrating an example of an apparatus 100 configured to automatically analyze cardiac images 102 and detect pathologies (e.g., medical abnormalities such as scars, myocardial edema, etc.) in the images based on one or more machine learning (ML) models. The cardiac images 102 may include, for example, cardiac magnetic resonance (CMR) images and/or tissue characterization maps, and the ML models may include an image classification model 104, a heart segmentation model 106, and/or a pathology detection model 108 (e.g., a machine learning or ML model may also be referred to herein as a machine-learned model, an artificial intelligence (AI) model, or a neural network model). In examples, the CMR images may include individual MR images of a heart or images from a cine movie of the heart, which may capture information regarding the healthy state of the heart in various directions (e.g., along a short-axis and/or a long-axis of the heart) and/or various parts (e.g., two chambers, three chambers, or four chambers) of the heart. The tissue characterization maps, on the other hand, may be generated based on various cardiac mapping techniques such as, e.g., T1 mapping, T2 mapping, etc.

The apparatus 100 may be configured to obtain the cardiac images 102 (e.g., a plurality of cardiac images) from one or more sources including, e.g., a magnetic resonance imaging (MRI) scanner and/or a medical record database. Upon obtaining these images, the apparatus 100 may be configured to automatically classify them into multiple groups based on the image classification model 104. For example, the apparatus 100 may be configured to classify the cardiac images 102 into at least a first group comprising one or more short-axis images of the heart and a second group comprising one or more long-axis images of the heart. The apparatus 100 may also be configured to classify the cardiac images 102 into a first group comprising two-chamber images, a second group comprising three-chamber images, and/or a third group comprising four-chamber images. The image classification model 104 may be trained to classify or categorize the cardiac images 102 based on various criteria, information, or characteristics of the images. For example, the image classification model 104 may be trained to determine the classification or category of a cardiac image 102 based on digital imaging and communications in medicine (DICOM) header information associated with the image, based on DICOM content of the image, and/or based on anatomical landmarks detected in the image. For instance, the image classification model 104 may be trained to detect a heart range in an image and determine whether a cardiac image 102 corresponds to a short-axis slice based on whether the cardiac image 102 contains the heart. This may be achieved, for example, by training the image classification model 104 to detect, from a long-axis image, anatomical landmarks such as the mitral annulus and/or apical tip, and use the detected anatomical landmarks to determine whether the cardiac image 102 contains the heart. This may also be achieved by comparing the cardiac image 102 with other CMR scans such as cine images known to be valid short-axis images.

All or a subset of the images classified based on the image classification model 104 (e.g., at least one group of cardiac images from the multiple groups categorized by the image classification model 104) may be further processed by the apparatus 100, for example, to determine the existence (or non-existence) of a cardiac pathology (e.g., a cardiac abnormality) and/or to calculate (and report) certain mechanical or electrical parameters (e.g., strain values) of the heart. The processing may include, for example, segmenting the heart in one or more cardiac images into multiple anatomical regions based on the heart segmentation model 106, such that the pathology detection and/or parameter reporting tasks described above may be performed at a region or segment level. For instance, the heart segmentation model 106 may be trained to segment the heart into 16 or 17 segments based on standards published by the American Heart Association (AHA) such that respective cardiac parameters associated with the segments (e.g., the average strain value or myocardial thickness associated with each of the segments) may be determined and reported (e.g., in the form of a bullseye plot). As another example, the heart segmentation model 106 may be trained to segment the heart based on chambers (e.g., such as the left ventricle (LV) and the right ventricle (RV)) and/or other anatomies (e.g., such as the papillary), and perform the pathology detection and/or parameter reporting tasks based on the segmentation. As will be described in greater detail below, the heart segmentation model 106 may be trained to conduct the segmentation operation on a cardiac image (e.g., which may be a CMR image or a tissue characterization map) by detecting anatomical landmarks in the cardiac image. The quality of the segmentation may be improved based on information extracted from and/or shared by other scans that may be associated with multiple spatial and/or temporal locations, different contrasts, etc.

The apparatus 100 may be configured to detect pathologies (e.g., including abnormal cardiac parameters) in one or more heart segments based on the pathology detection model 108. For instance, the pathology detection model 108 may be trained to learn visual features associated with an abnormal tissue pattern or property that may be indicative of a pathology (e.g., a hyper-intensity region on an LGE image may be linked to potential scars), and subsequently detect the abnormal tissue pattern or property in a cardiac image (e.g., an LGE image, a T1/T2 map, etc.) in response to detecting those visual features in the cardiac image. The pathology detection model 108 may be trained to make a comprehensive decision about a tissue pattern or property based on CMR images, tissue characterization maps, and/or images from other sequences such as T2-weighted images that may provide information regarding edema (e.g., a first-pass perfusion image may provide information regarding a microvascular flow to the myocardium, while a phase-contrast velocity encoded image may provide information regarding the velocity of the blood flow). Quantitative methods such as those based on signal thresholding may also be used to determine the tissue pattern or property. The apparatus 100 may be configured to indicate the detection of a pathology and/or the determination of a tissue pattern or property in various manners. For example, the apparatus 100 may indicate the detection of a medical abnormality by drawing a bounding box around the area of the cardiac image that contains the abnormality, or by segmenting the area that contains the abnormality from the cardiac image.

In examples, the cardiac images 102 may be captured at different time spots and/or characterized by different contrasts, and the apparatus 100 may be configured to align these cardiac images (e.g., in space) and process all or a subset of them together. For example, an ECV map may be generated based on a first T1 map obtained with contrast and a second T1 map obtained without contrast (e.g., by performing pixel-wise subtraction and/or division on the T1 maps), and the apparatus 100 may be configured to apply deep learning based techniques (e.g., using a pre-trained motion compensation model) to remove (e.g., filter out) the impact of patient breathing and/or motion from those T1 maps, thereby allowing elastic registration and accurate ECV estimation. The apparatus 100 may, for example, be configured to implement a first neural network and a second neural network that are trained (e.g., in a self-supervised manner) for registering the T1 map with contrast and the T1 map without contrast. The first neural network may be trained to register the two T1 maps and the second neural network may be trained to compensate for breathing motions and/or patient movements in either or both of the T1 maps. The second neural network may, for example, be trained to conduct a deformable image registration of the T1 maps to compensate for the motions or movements described herein. Using these techniques, contents of the T1 maps, which may be comparable despite the motions or movements, may be disentangled from the appearances (e.g., pixel-wise appearances) of the T1 maps, which may be un-comparable due to the motions or movements. In examples, the first and/or second neural network described above may utilize an encoder-decoder structure. The encoder may be used to encode a T1 map into a latent space comprising distinguishable appearance and content features, and the encoder may acquire the capability (e.g., through training) to ensure that similar content features are generated from the T1 map pair and that dissimilar features are represented by different appearances. The encoder and decoder networks may be trained with paired T1 maps, during which the networks may learn to utilize content features extracted from a pair of T1 maps to determine the similarity between the T1 maps.

The apparatus 100 may be configured to report the detection of cardiac pathologies and/or determination of cardiac parameters. The report may include, for example, numerical values, graphs, and/or charts. For instance, the apparatus 100 may be configured to determine respective parameters (e.g., strain values) and/or statistics associated with the AHA heart segments described herein, summarize the parameters and/or statistics into a bullseye plot, and include the plot in a report. As another example, the apparatus 100 may be configured to calculate and report, for one or more of the AHA heart segments, respective scar-to-normal tissue ratios, average T1 values, standard deviations of T1 values, and/or numbers of pixels having pixel values outside 3 standard deviations of an average. The apparatus 100 may also be configured to summarize one or more of the foregoing values into a global cardiac health score for a patient, and report the score for the patient. The summary may also be performed at a segment level, in which case a bullseye plot showing the respective summaries of multiple segments may be generated and included in a report.

In examples, the cardiac images 102 may include CMR images (e.g., from a cine movie) and tissue characterization maps (e.g., such as T1 and/or T2 maps) corresponding to the same underlying anatomical structure (e.g., the myocardium of the same patient), and one or more of the machine learning models described herein may be trained to utilize information obtained from the CMR images to supplement the automatic processing of the tissue characterization maps, or vice versa. For example, during the training of the heart segmentation model 104, cine images may be used to train the model first, and then transfer learning techniques such as fine-tuning may be applied based on tissue characterization maps to update the model parameters. As another example, the heart segmentation model 104 may be trained directly using both cine images and tissue characterization maps as inputs, and features extracted from one input (e.g., from the cine images) may be used to guide the segmentation of the other input (e.g., the tissue characterization maps), e.g., using an attention mechanism. As yet another example, the tissue characterization maps may be used indirectly (e.g., during pre- or post-processing operations) to improve the output of the machine learning models described herein. For instance, the heart segmentation model 104 may be trained using cine images to segment a myocardium. The cine images may then be registered with corresponding tissue characterization maps (or vice versa) before the tissue characterization maps are segmented to locate the myocardium. As yet another example, the heart segmentation model 104 may be trained to locate the intersection points of the LV and RV on cine images. These intersection points (e.g., or other landmark locations) may then be transferred to the tissue characterization maps and used to segment those tissue characterization maps. The transfer may be accomplished, for example, based on imaging parameters such as patient and/or imaging coordinates included in a DICOM header, and/or by registering the tissue characterization maps with the cine images.

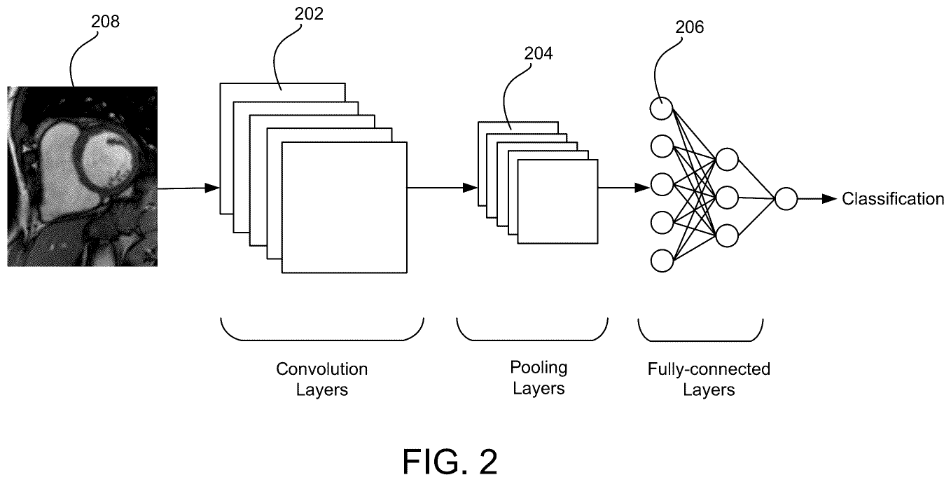

FIG. 2 illustrates an example of an artificial neural network (ANN) that may be used to implement and/or learn an image classification model such as the image classification model 104 described herein. As shown, the ANN may be a convolutional neural network (CNN) that may include a plurality of layers such as one or more convolution layers 202, one or more pooling layers 204, and/or one or more fully connected layers 206. Each of the convolution layers 202 may include a plurality of convolution kernels or filters configured to extract features from an input image 208 (e.g., a cine image). The convolution operations may be followed by batch normalization and/or linear (or non-linear) activation, and the features extracted by the convolution layers may be down-sampled through the pooling layers and/or the fully connected layers to reduce the redundancy and/or dimension of the features, so as to obtain a representation of the down-sampled features (e.g., in the form of a feature vector or feature map). Based on the feature representation, a classification prediction may be made, for example, at an output of the ANN to indicate whether the input image 208 is a short-axis image (e.g., a short-axis slice), a long-axis image (e.g., a long-axis slice), a 2-chamber image, a 3-chamber image, a 4-chamber image, etc. The classification may be indicated with a label (e.g., “short-axis image,” “long-axis image,” “2-chamber image”, etc.), a numeric value (e.g., 1 corresponding to a short-axis image, 2 corresponding to a long-axis image, 3 corresponding to a 2-chamber image, etc.), and/or the like.

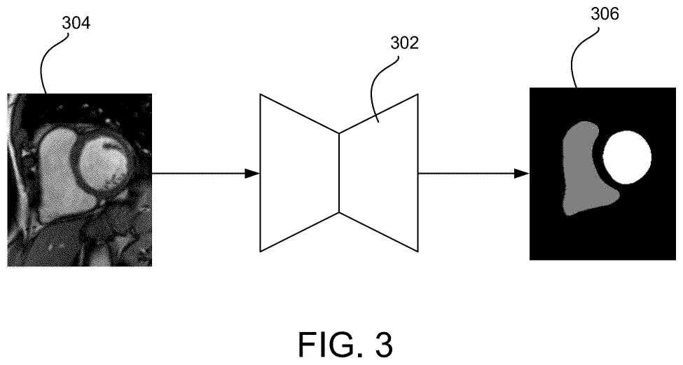

FIG. 3 illustrates an example of an artificial neural network (ANN) 302 that may be used to implement and/or learn an image segmentation model such as the heart segmentation model 106 described herein. The ANN 302 may utilize an architecture that includes an encoder network and a decoder network. The encoder network may be configured to receive an input image 304 such as a cine image, extract features from the input image, and generate a representation (e.g., a low-resolution or low-dimension representation) of the features at an output. The encoder network may be a convolutional neural network having multiple layers configured to extract and down-sample the features of the input image 304. For example, the encoder network may comprise one or more convolutional layers, one or more pooling layers, and/or one or more fully connected layers. Each of the convolutional layers may include a plurality of convolution kernels or filters configured to extract specific features from the input image. The convolution operation may be followed by batch normalization and/or non-linear activation, and the features extracted by the convolutional layers (e.g., in the form of one or more feature maps) may be down-sampled through the pooling layers and/or the fully connected layers to reduce the redundancy and/or dimension of the features. The feature representation produced by the encoder network may be in various forms including, for example, a feature map or a feature vector.

The decoder network of ANN 302 may be configured to receive the representation produced by the encoder network, decode the features of the input image 304 based on the representation, and generate a mask 306 (e.g., a pixel- or voxel-wise segmentation mask) for segmenting one or more objects (e.g., the LV and/or RV of a heart, the AHA heart segments, etc.) from the input image 302. The decoder network may also include a plurality of layers configured to perform up-sampling and/or transpose convolution (e.g., deconvolution) operations on the feature representation produced by the encoder network, and to recover spatial details of the input image 304. For instance, the decoder network may include one or more un-pooling layers and one or more convolutional layers. Through the un-pooling layers, the decoder network may up-sample the feature representation produced by the encoder network (e.g., based on pooled indices stored by the encoder network). The up-sampled representation may then be processed through the convolutional layers to produce one or more dense feature maps, before batch normalization is applied to the one or more dense feature maps to obtain a high dimensional representation of the input image 304. As described above, the output of the decoder network may include a segmentation mask for delineating one or more anatomical structures or regions from the input image 304. In examples, such a segmentation mask may correspond to a multi-class, pixel/voxel-wise probabilistic map in which pixels or voxels belonging to each of the multiple classes are assigned a high probability value indicating the classification of the pixels/voxels.

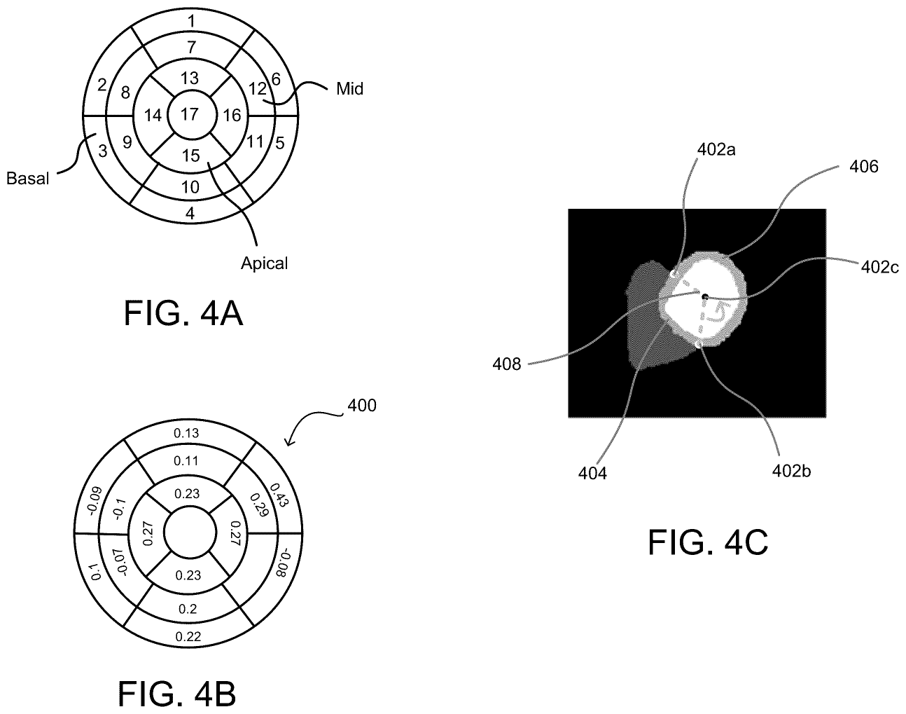

FIG. 4A illustrates examples of heart segments (e.g., segments of a myocardium) that may be identified from CMR images using the machine-learned heart segmentation model described herein (e.g., the heart segmentation 106). As shown, the heart segmentation model may be trained to identify and divide the myocardium of a human heart depicted in a CMR image into seventeen segments, subsets of which may respectively belong to a basal section of the myocardium, a middle (or mid) section of the myocardium, or an apical section of the myocardium. For example, the segments labeled 1-6 in the figure may belong to the basal section of the myocardium, with 1 representing the basal anterior, 2 representing the basal anteroseptal, 3 representing the basal inferoseptal, 4 representing the basal inferior, 5 representing the basal inferolateral, and 6 representing the basal anterolateral. The segments labeled 7-12 may belong to the middle section of the myocardium, with 7 representing the mid anterior, 8 representing the mid anteroseptal, 9 representing the mid inferoseptal, 10 representing the mid inferior, 11 representing the mid inferolateral, and 12 representing the mid anterolateral. The segments labeled 13-16 may belong to the apical section of the myocardium, with 13 representing the apical anterior, 14 representing the apical septal, 15 representing the apical inferior, and 16 representing the apical lateral. There may also be a 17th segment that corresponds to the apex of the heart (e.g., the apex of the myocardium). Based on these heart segments, cardiac tissue patterns, properties, and/or parameters may be determined and/or reported. For example, as shown in FIG. 4B, respective strain values (e.g., average strain values) may be calculated for the aforementioned heart segments and a bullseye plot 400 may be generated to report the strain values.

FIG. 4C shows an example of segmenting a myocardium into the segments shown in FIG. 4A based on anatomical landmarks detected in an CMR image or tissue characterization map. These anatomical landmarks may include, for example, landmarks 402a and 402b, which may represent locations or points where the RV intersects the LV (e.g., at the anterior and inferior LV myocardium), and landmark 402c, which may represent a center of the LV. Landmarks 402a and 402b may divide the myocardium into two parts, 404 and 406, where part 404 may be shorter than part 406. The basal and mid-section segments of the myocardium (e.g., corresponding to segments 1-6 and segments 7-12 of FIG. 4A) may then be obtained by dividing (e.g., equally) the shorter part 404 into two segments and the longer part 406 into four segments, while the apical segments of the myocardium (e.g., corresponding to segments 13-16 of FIG. 4A) may be obtained by treating part 404 as one myocardial segment and dividing (e.g., equally) part 406 into another three segments. For instance, one or more of the aforementioned heart segments may be obtained by drawing an imaginary line 408 between landmark 402c and either of landmarks 402a and 402b, and rotating this imaginary line at equal angular steps around the myocardium. The angular steps may be, for example, 60 degrees if the myocardium is to be divided into six segments (e.g., for the basal section and the middle section) and 90 degrees if the myocardium is to be divided into four segments (e.g., for the apical section).

The anatomical landmarks described above (e.g., such as the landmarks 402a and 402b) may be detected using a landmark detection neural network (e.g., a machining learning model implemented by the neural network). In examples, such a neural network may include an CNN (e.g., having one or more the convolutional, pooling, and/or fully-connected layers described herein), which may be trained to learn features associated with the anatomical landmarks from a training dataset (e.g., comprising CMR images, tissue characterization maps, and/or segmentation masks), and subsequently determine the anatomical landmarks in a segmentation mask, an CMR image, or a tissue characterization map in response to detecting those features.

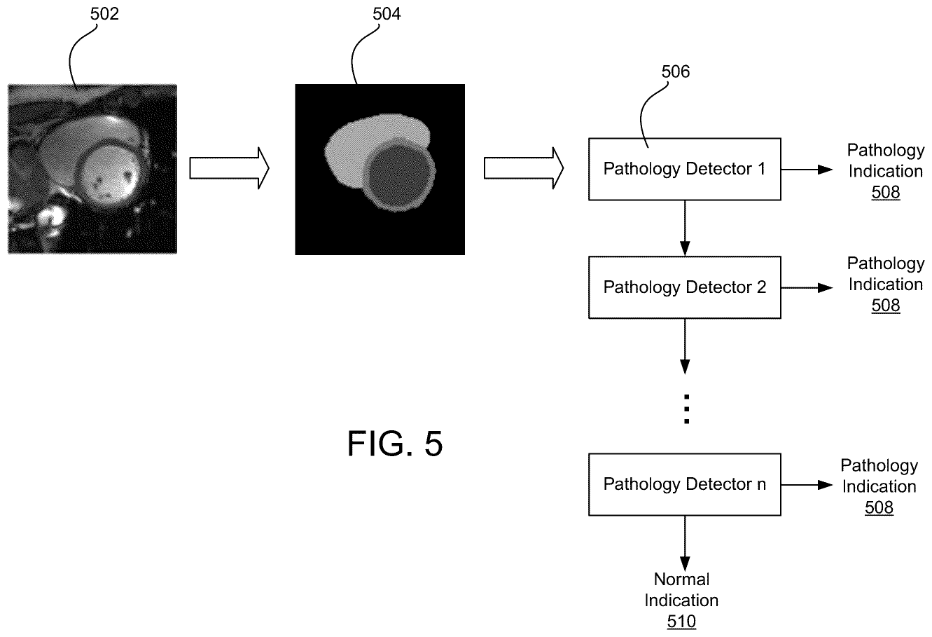

FIG. 5 illustrates an example of automatically determining a cardiac pathology using a machine learning model (e.g., the pathology detection model 108 of FIG. 1). As shown, the pathology may be determined in a cardiac image 502, which may be a cine image or a tissue characterization map (e.g., a T1 map). The cardiac image 502 may be segmented (e.g., a segmentation mask 504 may be generated), for example, using the heart segmentation model 106 of FIG. 1, such that one or more anatomical regions of interest may be delineated in the cardiac image 502. The segmented image may then be further processed through one or more pathology detectors 506 to detect the existence (or non-existence) of a pathology. In some implementations, the one or more pathology detectors 506 may be implemented and/or learned using a respective neural network, while in other implementations, multiple pathology detectors 506 may be implemented and/or learned using a same neural network. As shown in FIG. 5, the cardiac image 502 and/or the segmentation mask 504 may be processed (e.g., sequentially) through multiple pathology detectors 506, each of which may be trained to detect a specific pathology (e.g., a medical abnormality) including, e.g., a scar tissue, an RV abnormality (RVA), a hypertrophic cardiomyopathy (HCM), a dilated cardiomyopathy (DCM), a myocardial infarction (MIFN), etc. Each time a pathology is detected, a corresponding indication 508 (e.g., a label, a bounding box, a mask, etc.) may be provided to indicate the detection. If no pathology is detected by any pathology detector 506, an indication 510 may be provided to indicate that the pathological condition of the corresponding region of interest is normal.

In examples, each pathology detector 506 may be trained to learn visual features associated with one or more specific pathologies (e.g., a hyper-intensity region on an LGE image may be linked to potential scars) based on a training dataset (e.g., cardiac images containing the pathology), and subsequently determine that the one or more pathologies are presented in the cardiac image 502 in response to detecting those visual features in a region or area of the cardiac image 502. In examples, one or more of the pathology detectors 506 may be trained to determine structural and/or kinematic information of the heart, and calculate cardiac parameters (e.g., strain values) associated with a region or segment of the heart based on the structural and/or kinetic information. For instance, one or more of the pathology detectors 506 may be trained to process multiple cardiac images 502 (e.g., from a cine movie) and determine the motion of the myocardium by extracting respective features from a first cardiac image and a second cardiac image (e.g., any two images in the cine movie). The one or more pathology detectors 506 may then identify changes between the two sets of features, and generate a motion field that represents the changes. By repeating these operations for other images of the cine movie, the one or more pathology detectors 506 may track the motion of the myocardium throughout a cardiac cycle and calculate the myocardial strains of the heart (e.g., pixel-wise strain values), for example, by conducting a finite strain analysis of the myocardium (e.g., using one or more displacement gradient tensors calculated from the motion fields). In some embodiments, the one or more pathology detectors 506 may determine a respective aggregated strain value for each of the regions of interest (e.g., by calculating an average of the pixel-wise strain values in each region) and report the aggregated strain values, for example, via a bullseye plot (e.g., the bullseye plot 400 in FIG. 4B).

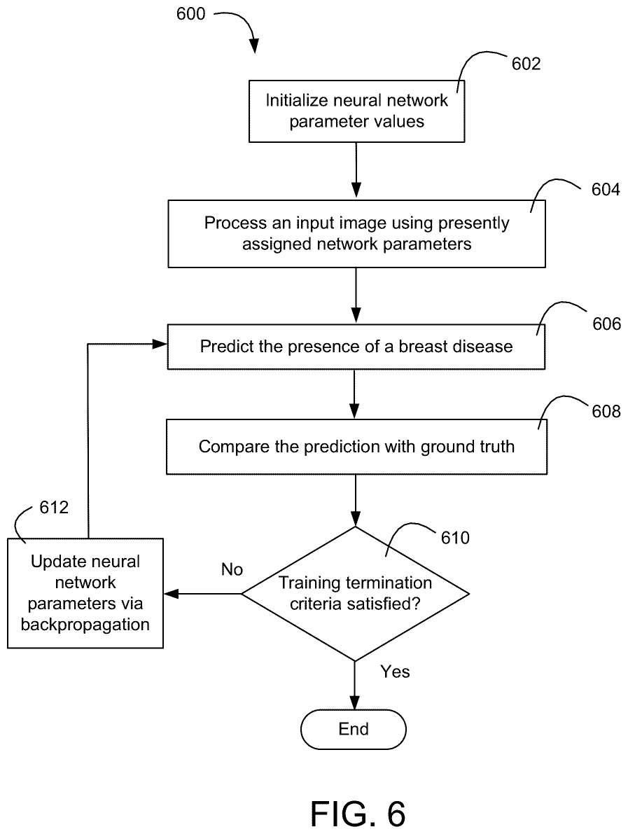

FIG. 6 shows a flow diagram illustrating an example process 600 for training a neural network (e.g., an ML model implemented by the neural network) to perform one or more of the tasks described herein. As shown, the training process 600 may include initializing the operating parameters of the neural network (e.g., weights associated with various layers of the neural network) at 602, for example, by sampling from a probability distribution or by copying the parameters of another neural network having a similar structure. The training process 600 may further include processing an input training image (e.g., a cine image or tissue characterization map) using presently assigned parameters of the neural network at 604, and making a prediction about a desired result (e.g., a classification label, a segmentation mask, a pathology detection, etc.) at 606. The prediction result may be compared, at 608, to a ground truth, and a loss associated with the prediction may be determined based on the comparison and a loss function. The loss function employed for the training may be selected based on the specific task that the neural network is trained to do. For example, if the task involves a classification or segmentation of the input image, a loss function based on a mean squared error between the prediction result and the ground truth may be used, and if the task involves detecting the location of an pathology (e.g., by drawing a bounding box around the pathology), a loss function based on generalized intersection over union (GIOU) may be used.

At 610, the loss calculated using one or more of the techniques described above may be used to determine whether one or more training termination criteria are satisfied. For example, the training termination criteria may be determined to be satisfied if the loss is below a threshold value or if the change in the loss between two training iterations falls below a threshold value. If the determination at 610 is that the termination criteria are satisfied, the training may end; otherwise, the presently assigned network parameters may be adjusted at 612, for example, by backpropagating a gradient descent of the loss function through the network before the training returns to 606.

For simplicity of explanation, the training steps are depicted and described herein with a specific order. It should be appreciated, however, that the training operations may occur in various orders, concurrently, and/or with other operations not presented or described herein. Furthermore, it should be noted that not all operations that may be included in the training method are depicted and described herein, and not all illustrated operations are required to be performed.

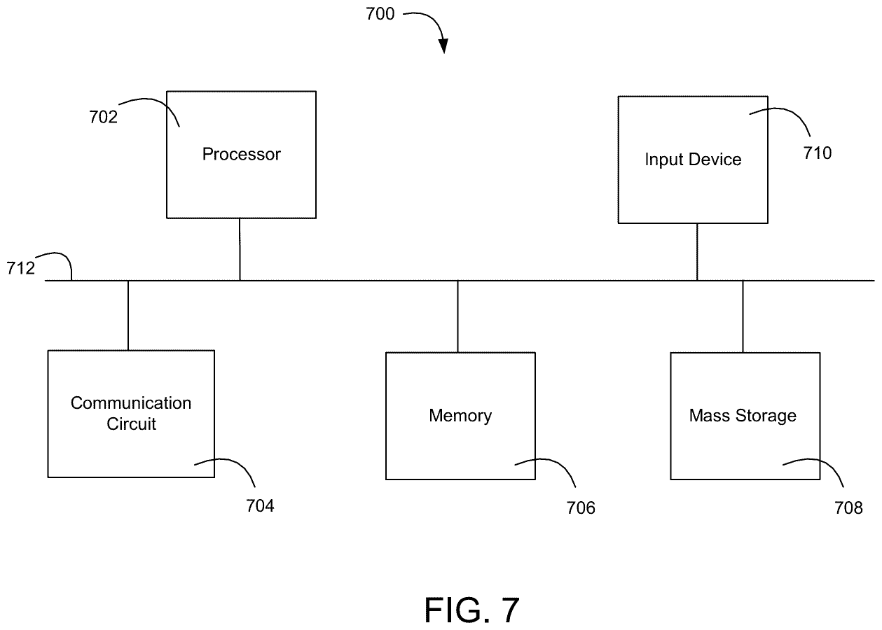

The systems, methods, and/or instrumentalities described herein may be implemented using one or more processors, one or more storage devices, and/or other suitable accessory devices such as display devices, communication devices, input/output devices, etc. FIG. 7 illustrates an example of an apparatus 700 that may be configured to perform the tasks described herein. As shown, apparatus 700 may include a processor (e.g., one or more processors) 702, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 700 may further include a communication circuit 704, a memory 706, a mass storage device 708, an input device 710, and/or a communication link 712 (e.g., a communication bus) over which the one or more components shown in the figure may exchange information.

Communication circuit 704 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 706 may include a storage medium (e.g., a non-transitory storage medium) configured to store machine-readable instructions that, when executed, cause processor 702 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 708 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 702. Input device 710 may include a keyboard, a mouse, a voice-controlled input device, a touch sensitive input device (e.g., a touch screen), and/or the like for receiving user inputs to apparatus 700.

It should be noted that apparatus 700 may operate as a standalone device or may be connected (e.g., networked or clustered) with other computation devices to perform the tasks described herein. And even though only one instance of each component is shown in FIG. 7, a skilled person in the art will understand that apparatus 700 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description.

What is claimed is:

1. An apparatus, comprising:
at least one processor configured to:
obtain a plurality of medical images associated with a heart;
classify, based on a machine-learned image classification model, the plurality of medical images into multiple groups, wherein the multiple groups include at least a first group comprising one or more short-axis images of the heart and a second group comprising one or more long-axis images of the heart; and
process at least one group of medical images from the multiple groups, wherein, during the processing, the at least one processor is configured to:
segment, based on a machine-learned heart segmentation model, the heart in one or more medical images into multiple anatomical regions;
determine whether a medical abnormality exists in at least one of the multiple anatomical regions; and
provide an indication of the determination.

2. The apparatus of claim 1, wherein the plurality of medical images of the heart includes at least one of a magnetic resonance (MR) image of the heart or a tissue characterization map of the heart.

3. The apparatus of claim 1, wherein the at least one processor being configured to classify the plurality of medical images into the multiple groups comprises the at least one processor being configured to detect, based on the machine-learned image classification model, one or more anatomical landmarks associated with a short axis of the heart in a subset of the plurality of medical images, and classify the subset of medical images as belonging to the first group.

4. The apparatus of claim 3, wherein the one or more anatomical landmarks include at least one of an mitral annulus or an apical tip.

5. The apparatus of claim 1, wherein the one or more anatomical regions include a left ventricle of the heart and a right ventricle of the heart.

6. The apparatus of claim 1, wherein the one or more anatomical regions include multiple myocardial segments comprising one or more basal segments, one or more mid-cavity segments, and one or more apical segments.

7. The apparatus of claim 5, wherein the machine-learned heart segmentation model is trained to detect, in the at least one group of medical images, one or more anatomical landmarks that indicate where a left ventricle of the heart intersects with a right ventricle of the heart, and segment the heart into the multiple myocardial segments based on the one or more anatomical landmarks.

8. The apparatus of claim 1, wherein the at least one processor being configured to determine whether the medical abnormality exists in the at least one of the multiple anatomical regions comprises the at least one processor being configured to determine a tissue pattern or tissue parameter associated with the at least one of the multiple anatomical regions, and determine whether the medical abnormality exists in the at least one of the multiple anatomical regions based on the determined tissue pattern or tissue parameter.

9. The apparatus of claim 8, wherein the tissue pattern or tissue parameter is determined based on a machine-learned pathology detection model trained for determining the tissue pattern or tissue parameter, the machine-learned pathology detection model further trained to segment an area of the heart that is associated with the tissue pattern or tissue parameter from the at least one of the multiple anatomical regions.

10. The apparatus of claim 1, wherein the at least one processor is further configured to register two or more of the plurality of medical images based on a machine-learned image registration model, the image registration model trained to compensate for a motion associated with the two or more medical images during the registration.

11. The apparatus of claim 1, wherein the indication comprises a segmentation of the medical abnormality or a report of the medical abnormality.

12. A method of processing cardiac images, the method comprising:
obtaining a plurality of medical images associated with a heart;
classifying, based on a machine-learned image classification model, the plurality of medical images into multiple groups, wherein the multiple groups include at least a first group comprising one or more short-axis images of the heart and a second group comprising one or more long-axis images of the heart; and
processing at least one group of medical images from the multiple groups, wherein the processing comprises:
segmenting, based on a machine-learned heart segmentation model, the heart in one or more medical images into multiple anatomical regions;
determining whether a medical abnormality exists in at least one of the multiple anatomical regions; and
providing an indication of the determination.

13. The method of claim 12, wherein the plurality of medical images of the heart includes at least one of a magnetic resonance (MR) image of the heart or a tissue characterization map of the heart.

14. The method of claim 12, wherein classifying the plurality of medical images into the multiple groups comprises detecting, based on the machine-learned image classification model, one or more anatomical landmarks associated with a short axis of the heart in a subset of the plurality of medical images, and classifying the subset of medical images as belonging to the first group.

15. The method of claim 12, wherein the one or more anatomical regions include a left ventricle of the heart and a right ventricle of the heart.

16. The method of claim 12, wherein the one or more anatomical regions include multiple myocardial segments comprising one or more basal segments, one or more mid-cavity segments, and one or more apical segments.

17. The method of claim 16, wherein the machine-learned heart segmentation model is trained to detect, in the at least one group of medical images, one or more anatomical landmarks that indicate where a left ventricle of the heart intersects with a right ventricle of the heart, and segment the heart into the multiple myocardial segments based on the one or more anatomical landmarks.

18. The method of claim 12, wherein determining whether the medical abnormality exists in the at least one of the multiple anatomical regions comprises determining a tissue pattern or tissue parameter associated with the at least one of the multiple anatomical regions, and determining whether the medical abnormality exists in the at least one of the multiple anatomical regions based on the determined tissue pattern or tissue parameter.

19. The method of claim 18, wherein the tissue pattern or tissue parameter is determined based on a machine-learned pathology detection model trained for determining the tissue pattern or tissue parameter, the machine-learned pathology detection model further trained to segment an area of the heart that is associated with the tissue pattern or tissue parameter from the at least one of the multiple anatomical regions.

20. The method of claim 12, further comprising registering two or more of the plurality of medical images based on a machine-learned image registration model, the image registration model trained to compensate for a motion associated with the two or more medical images during the registration.
